(19) United States
(12) Patent Application Publication
(10) Pub. No.: US 2015/0037817 A1
(43) Pub. Date: Feb. 5, 2015

(54) BIFUNCTIONAL TUMOR DIAGNOSIS REAGENT AND METHOD FOR TUMOR DIAGNOSIS

(71) Applicant: INSTITUTE OF BIOPHYSICS, CHINESE ACADEMY OF SCIENCE, Beijing (CN)

(72) Inventors: Xiyun Yan, Beijing (CN); Minmin Liang, Beijing (CN); Kelong Fan, Beijing (CN); Dongling Yang, Beijing (CN); Di Lu, Beijing (CN); Jing Feng, Beijing (CN); Demin Duan, Beijing (CN)

(21) Appl. No.: 14/117,229

(22) PCT Filed: May 10, 2012
(86) PCT No.: PCT/CN12/75291

§ 371 Date: Oct. 24, 2014

(30) Foreign Application Priority Data
May 12, 2011 (CN) 201110122433.0

(51) Int. Cl. G01N 33/574

(52) U.S. Cl. CPC G01N 33/574; G01N 2800/7028
USPC 435/7.8

(57) ABSTRACT

The invention relates to a bifunctional tumor diagnostic reagent and a method for tumor diagnosis. The reagent consists of a protein shell specifically recognizing a cancer tissue and/or a cancer cell and an inorganic nano-core having the catalytic activity of a peroxidase. The bifunctional tumor diagnostic reagent has two functions, i.e., tumor specific identification and color development, and enables the tumor specific identification and the color development to be completed in one step, and the operation of the process is simple and convenient.

### TECHNICAL FIELD

The present invention falls into an interdisciplinary field covering nano-technology, bionics, immunology and biomedicine. In particular, the present invention provides a tumor diagnostic reagent which integrates two functions, i.e., tumor specific identification and visualization. The present invention also provides a method for the diagnosis of cancer tissues and cancer cells using the said tumor diagnostic reagent.

### BACKGROUND

Malignant tumor has become one of the common life-threatening diseases. Pathological section identification is one of the most precise and reliable method, which is recognized as the “Golden Standard” for tumor diagnosis in home and abroad (Shi, et al., (2008) Am. J. Clin. Pathol., 129:358-366; Taylor et al. (2006) Biotech Histochem., 81:3-12.; Larsson, et al., (1988) Immunocytochemistry: Theory and Practice. Boca Raton, Fla.: CRC Press 41-73). At present, the main staining method for a pathological section includes haematoxylin-eosin (HE) staining, immunohistochemistry and immunofluorescence. In HE staining, the chromatin in the nuclei is stained royal blue with haematoxylin, and the cytoplasm and the components in the extracellular matrix are stained red with eosin, so that the information about cell morphology is provided. As a result, based on the change of the cell morphology, a pathological physician can make a rough identification for the carcinogenesis of tissues and cells. Such a staining method is convenient and quick in manipulation, but it can only provide the change of cell morphology. With such relatively less information, it is not possible to make a precise judgment for more complicated and polytype tumors. In the immunohistochemistry or immunofluorescence method, a primary antibody binds to an antigen in the tissue to be detected, and then a fluorescent signal molecule-labeled or an enzyme-labeled secondary antibody or tertiary antibody binds to the primary antibody; subsequently, information about the location and semi-quantification of the antigen to be detected is provided by a fluorescent signal or color development through enzymatic substrate reaction, and pathological changes such as carcinogenesis, necrosis of the tissues and cells, and the infiltration of inflammatory cells and the like are further identified. The immunohistochemistry or immunofluorescence can provide detailed information about the distribution and amount of a specific antigen and the cell morphology, and is very helpful for thorough research of the pathology. However, the immune-staining requires several steps of incubation with the primary antibody, the secondary antibody, or even the tertiary antibody, repeated washings with PBS, labeling with an enzyme or a fluorescent molecule, which involves complex steps and a long processing time. Therefore, in order to improve the clinically pathological diagnosis efficiency and win time for the treatment of patients, it is necessary to investigate a reagent and a simple and quick method which is capable of providing abundant information for detecting cancerous tissues and cancerous cells.

Natural human ferritin is a spherical protein for iron storage, which is assembled from 24 heavy chain or light chain subunits at any ratio. The heavy chain subunit and the light chain subunit are highly homologous, and have a molecular weight of 21 kDa and 19 kDa, respectively (Theil, (1987) Annu. Rev. Biochem., 56:289-315). Ferritin has different subunit components in different human organs and tissues, with heavy chain subunits predominant in heart, and light chain subunits predominant in liver. The two types of subunits vary with the environmental requirements, which provides flexible supply of iron ions to the organism. However, only the heavy chain subunit is capable of converting Fe²⁺ to Fe³⁺ using oxygen so as to facilitate iron ions to successfully enter ferritin. Therefore, increasing the heavy chain subunit components can improve the cell's availability of iron, and increasing the light chain subunit components can improve the efficiency of iron storage. In the spherical shell of ferritin, iron ions form a crystalline with phosphates and hydroxyl ions, which has similar chemical properties to iron oxide hydrate in the minerals (Harrison, et al., (1996) Biochim. Biophys. Acta 1275:161-203; Levi, et al. (1988) J. Biol. Chem. 263:18086-18092; Ford, et al. (1984) Philos Trans. R. Soc. Lond B Biol. Sci. 304:551-565).

In addition to storing iron in cells, heavy chain subunit of ferritin is capable of specifically binding to activated lymphocytes, leukemic cells, cervical cancer cells, lymphoma cells, and the like (Fargion, et al. (1988) Blood 71:753-757; Moss, et al. (1992) J. Lab Clin. Med 119:273; Bretscher, et al. (1983) EMBO J. 2:599-603; Takami, et al. (1986) Biochim. Biophys Acta 884:31-38; Fargion, et al. (1991) Blood, 78:1056-1061). Li et al. reported that transferrin receptor 1 (TfR1) on the cell surface, as a co-receptor of the heavy chain subunit of ferritin, mediates the interaction between ferritin and cells (Li, et al., (2010) Proc. Natl. Acad Sci. USA 107: 3505-3510). The transferrin receptor is a critical molecule for the cell to obtain iron element, and is essential for cell proliferation and rapid growth of tumor cells. Many tumors such as hepatocellular carcinoma, breast cancer, pancreatic cancer, stomach cancer, colon cancer, and the like have a high expression level of TfR1, so that TfR1 is a specific target for identification and treatment of tumors (Larrick, et al. (1979) J. Supramol. Struct. 11:579-586; Daniels, et al. (2006) Clin. Immunol., 121:144-158; Ryschich, et al. (2004) Eur. J. Caner 40:1418-1422.). Since TfR1 is a co-receptor of the heavy chain subunit of ferritin, the heavy chain subunit of ferritin can be used to specifically identify tumors.

The natural protein shells having various sizes of nano-cavity structures mainly comprise apoferritin, heat shock protein (HSP, with a diameter of 12 nm), DNA-binding protein (DPs, with a diameter of 9 nm), pteridine synthase (LS, with a diameter of 15 nm) and viral protein shells such as cowpea chlorotic mottle virus (CCMV, with a diameter of 28 nm) and cowpea mosaic virus protein (CMV, with a diameter of 31 nm). Such a nano-cavity provides a good spatial structure for growing nanoparticles and efficiently preventing the aggregation of nanoparticles during synthesis. In view of such a specific structure, inorganic particles of various sizes can be synthesized in the cavity, and a controlled synthesis of nano-materials in different sizes, shapes and crystalline structures can be achieved (Turyanska L., et al. (2009) Small, 5:1738-1741; Meldrum F C., et al. (2009) Science, 257:522-523; Galvez N., et al. (2006) J. Mater. Chem., 16:2757-2761; Hennequin B., et al. (2008) Adv. Mater., 20:3592-3593; Yamashita I., et al. (2004) Chem. Lett., 33:1158-1159). Moreover, these protein shells having different structures can be easily chemically coupled or genetically fused to target molecules and signal molecules so as to enable a targeted identification and detection of cancer. For example, apoferritin enables a targeted identification of cancer cells by genetically fused to RGD (Uchida M., et al. (2006) J. Am. Chem. Soc., 128: 16626-16633), and enables in vivo tumor imagining by coupled to chemical label molecule Cy5.5 or nano-cavity loaded isotope ⁶⁴Cu and gadolinium (Lin X., et al. (2011) Nano Lett., 11: 814-819; Geninatti C S., et al. (2006) Cancer Res., 66: 9196-201.).

In 2007, our team found that iron oxide nanoparticles have a similar catalytic activity to peroxidases for the first time, i.e., in the presence of hydrogen peroxide, iron oxide nanoparticles can react with the substrates of horseradish peroxidase such as DAB and TMB to generate the same reaction products as peroxidase does. So, iron oxide nanoparticles have a similar catalytic activity to peroxidases. Based on the finding, we proposed the concept of iron oxide nanoparticle enzyme mimic (Gao L, et al., (2007) Nature Nanotech., 2:577-583; Chinese Patent Application No. 200610057413.9).

### DESCRIPTION OF THE INVENTION

The present invention provides a novel tumor diagnostic reagent and a method for tumor diagnosis based on a nanoscale material having a peroxidase activity in combination with a tumor-recognizing protein.

The tumor diagnostic reagent of the present invention consists of a protein shell and a nanoparticle core, wherein the protein shell is capable of specifically recognizing cancerous tissues and cells, and the nanoparticle core has the catalytic activity of a peroxidase, that is, it is capable of reacting with the substrate of horseradish peroxidase (HRP) for color development. The tumor diagnostic reagent of the present invention is capable of specifically recognizing tissues and color-developing in one step, without the need of multiple steps of incubation with a primary antibody, a secondary antibody and a tertiary antibody, and of antibody-labeling with enzyme or signal molecule, as used in the conventional immunological methods. The experimental operation of the process is simple, convenient and quick.

Therefore, in an aspect, the present invention provides a bifunctional tumor diagnostic reagent, characterized in that it consists of a protein shell which specifically recognizes cancer tissues and/or cancer cells and an inorganic nano-core which has the catalytic activity of a peroxidase.

The protein shell specifically recognizing cancer tissues and/or cancer cells according to the present invention may be selected from various known proteins specifically recognizing cancer tissues and/or cancer cells in the art. In some embodiments, the protein shell of the present invention is a genetically recombinant or natural apoferritin. In some embodiments, the apoferritin may be self-assembled from 12 or 24 heavy chain subunits and light chain subunits at any ratio. In the present invention, the natural apoferritin may be derived from an eukaryotic or a prokaryotic organism, preferably from a mammal. In some embodiments, the protein shell of the present invention is a genetically recombinant ferritin consisting of only heavy chain subunits.

In some embodiments, the protein shell of the present invention is heat shock protein (HSP, with a diameter of 12 nm), DNA-binding protein (DPs, with a diameter of 9 nm), pteridine synthase (LS, with a diameter of 15 nm) or a viral protein shell comprising cowpea chlorotic mottle virus (CCMV, with a diameter of 28 nm) and cowpea mosaic virus protein (CMV, with a diameter of 31 nm).

In some embodiments, the protein shell of the present invention is chemically coupled or genetically fused to a target molecule and/or a signal molecule. In some of such embodiments, the target molecule according to the present invention comprises polypeptide or nucleic acid aptamer. In some of such embodiments, the target molecule according to the present invention is an antibody. In some embodiments, the signal molecule according to the present invention comprises fluorescence and radioactive nuclide.

In some embodiments, the inorganic nano-core of the present invention has the catalytic activity of a peroxidase, to react with substrates for color-development, which comprises, but is not limited to, magnetic iron oxide nanoparticles, iron sulfide nanoparticles, noble metal-blended iron oxide nanoparticles, double-metal alloy nanoparticles, or cerium oxide nanoparticles.

In some embodiments, the protein shell of the present invention is a genetically ferritin, wherein all the subunits of the ferritin are heavy chain subunits. In some embodiments, the inorganic core of the present invention is a magnetic iron oxide nanoparticle.

In another aspect, the present invention provides a kit for tumor diagnosis, comprising said bifunctional tumor diagnostic reagent of the present invention. In some embodiments, the kit further comprises a peroxidase substrate. In some embodiments, the peroxidase substrate comprises DAB and TMB. In some embodiments, the kit of the present invention further comprises an instruction.

In a further aspect, the present invention provides use of the bifunctional tumor diagnostic reagent of the present invention in the manufacture of a tumor diagnostic reagent. As described above, the bifunctional tumor diagnostic reagent of the present invention consists of a protein shell and a nanoparticle core, wherein the protein shell is capable of specifically recognizing cancerous tissues and cells, and the nanoparticle core has the catalytic activity of a peroxidase to react with the substrate of horseradish peroxidase for color development. Therefore, it can be used for tumor diagnosis.

In another aspect, the present invention provides a bionic protein, which consists of a protein shell specifically recognizing cancer tissues and/or cancer cells and an inorganic nano-core having the catalytic activity of a peroxidase. In some embodiments, the bionic protein of the present invention is bionic ferritin. In some embodiments, the protein shell of the present invention is a genetically recombinant or natural apoferritin. In some embodiments, the apoferritin may be self-assembled from 12 or 24 heavy chain subunits and light chain subunits at any ratio. In some embodiments, the natural apoferritin may be derived from an eukaryotic or a prokaryotic organism, preferably from a mammal. In some embodiments, the protein shell of the present invention is a genetically recombinant ferritin consisting of only heavy chain subunits. The present invention also provides use of said bionic protein in the manufacture of an agent for tumor detection. As described above, the bionic protein of the present invention is composed of a protein shell and a nanoparticle core, wherein the protein shell is capable of specifically recognizing cancer tissues and cells, and the nanoparticle core has the catalytic activity of a peroxidase to react with HRP substrate for color-development. Therefore, it can be used for tumor diagnosis.

In a further aspect, the present invention provides a method for detecting a tumor in an individual, comprising: obtaining a body tissue or cell sample from said individual, contacting said sample with the bifunctional tumor diagnostic reagent of the present invention or the bionic protein of the present invention, adding a substrate for direct development, thereby determining the presence of the tumor in said individual. In some embodiments, said tissue is the puncture biopsy specimens, postoperative pathological sections or autopsy tissues from the clinical patients or animal models. In some embodiments, said cell is exfoliated cells in the circulatory system of organism, tissue lytic cells or in vitro cultured cells.

The reagent and method of the present invention can be used for clinical cancer screening, early-stage diagnosis, diagnosis for cancer cells' metastasis, monitoring a treatment, tumor imagining, pathological analysis of autopsy tissues or postoperative recurrence evaluation. The tumor diagnostic reagent provided by the present invention can accomplish tissue-specific recognition and color-development in one step, without the need of multiple steps of incubation with a primary antibody, a secondary antibody and a tertiary antibody labeled with enzyme or signal molecule, as used in conventional immunological methods. The manipulation process is simple, convenient and quick.

### THE DESCRIPTION OF DRAWINGS

Now the preferred embodiments of the present invention are described by referring to the following drawings:

FIG. 1: A). the TEM photos of the bionic ferritin; B). the bionic ferritin has the catalytic activity of a peroxidase to catalyze HRP substrate, TMB, for color development; C). the bionic ferritin has the catalytic activity of a peroxidase to catalyze HRP substrate, DAB, for color development.

FIG. 2: the analysis of the interaction of colon rectal cancer cells HT29 with the bionic ferritin by flow cytometry. A). the competitive binding of the cell surface receptors; a: PBS control, b: staining with Cy5.5 labeled-bionic ferritin, c: the cell surface receptors are blocked with excessive un-labeled bionic ferritin; B). excessive anti-transferrin receptor 1 (TfR1) antibody competitively binds to the site of the bionic ferritin receptor, a: PBS control, b: Cy5.5 labeled-bionic ferritin, c: the sites of the receptor are blocked with excessive anti-TfR1 antibody; C). the cell-binding saturation curve of the bionic protein.

FIG. 3: the bionic ferritin specifically recognizes implanted tumor in mice. The staining region of the tumor tissue and the staining effect are comparable with those of an antibody (scale: 100 μm).

FIG. 4: the bionic ferritin specifically recognizes human hepatocellular carcinoma tissue sections, and does not recognize normal liver tissue, hepatitis or cirrhosis tissues. A)-D). four cases of hepatocellular carcinoma; E). normal liver tissue; F). the chronic inflammation of liver tissues; G). nodular cirrhosis; H). biliary cirrhosis; I). hepatocellular carcinoma; J). normal liver tissue; K). hepatocellular carcinoma; L). the chronic inflammation of liver tissues (scale: 100 μm).

FIG. 5: the staining of various human cancerous tissues and normal tissues with the bionic ferritin. A1). colon cancer, stage II; A2). colon mucous membrane tissue; B1). mammary non-specific infiltrative duct carcinoma, stage I; B2). breast fibroadenosis; C1). ovary papillary serous adenocarcinoma, stage III; C2). ovary tissue; D1). prostate cancer, stage 4; D2). prostate smooth muscle tissue; E1). pulmonary squamous cell carcinoma, stage III; E2). pulmonary tissues companied with congestion; F1). esophagus cell cancer, stage III; F2). chronic esophagus inflammation companied with epithelial proliferation; G1). thymic carcinoma; G2). thymic tissue; H1). cervix squamous cell carcinoma, stage III; H2)., chronic cervix inflammation.

FIG. 6: Fluorescence-labeled bionic ferritin shell specifically recognizes various human cancerous tissues. A1). colon cancer, stage III; A2). chronic inflammation of colon mucous membrane companied with epithelial proliferation; B1). mammary non-specific infiltrative duct carcinoma, stage I; B2). breast tissue; C1). ovary papillary serous adenocarcinoma, stage III; C2). ovary tissue; D1). colon adenocarcinoma, stage II; D2). colon mucous membrane tissue; E1). prostate cancer, stage 4; E2). prostate proliferation companied with PIN, stage I; F1). pulmonary squamous cell carcinoma, stage III; F2). pulmonary tissues; G1). ovary papillary serous adenocarcinoma, stage III; G2). ovary tissue; H1). pulmonary squamous cell carcinoma, stage III; H2). pulmonary tissues companied with congestion; I1). esophagus squamous cell cancer, stage III; I2). chronic esophagus inflammation companied with epithelial proliferation; J1). thymic carcinoma; J2). thymic tissue; K1). cervix squamous cell carcinoma, stage II; K2). cervix tissue; L1). cervix squamous cell carcinoma, stage III; L2). chronic cervix inflammation (scale: 50 μm).

FIG. 7: the map of the plasmid used in the present invention.

### EXAMPLES

The following examples are provided only for the purpose of illustration, and are not intended to limit the scope of the present invention in any way.

### Example 1

### The Preparation of the Bionic Ferritin

The protein shell consisting of only the full heavy chain of human ferritin was obtained by a genetic recombination method. The iron ions were trans-loaded into the protein shell by the means of human biomineralization, and then the loaded iron ions were oxidized to iron oxide nanoparticles. We bionically synthesized a human ferritin which consists of only the full heavy chain of human ferritin and which encloses a magnetic iron oxide nano-core. The protein is named the bionic ferritin and it was used for tumor diagnosis.

The specific protocols were as follows:

At first, we constructed the recombinant plasmid pET12b-HFn containing the gene sequence which encodes only heavy chain subunit of human ferritin (Santambrogio P. et al., (2000), Protein Expr. Purif., 19: 212-218). The whole cDNA library of human skeletal muscle was purchased from Invitrogen (D8090-01, Carlsbad, Calif., USA). The gene encoding only the heavy chain of human ferritin was isolated and amplified from the cDNA library via PCR. The two primers were designed as follows: forward PCR primer: 5′-A GTC GCC CAT ATG ACG ACC GCG TCC-3′ (Nde I site was underlined), with seven protection bases for enzyme digestion added, reverse PCR primer: 5′-GCC GGA TCC TTA GCT TTC ATT ATC AC-3′ (BamHI site was underlined), with three protection bases for enzyme digestion added. 552 bp amplified gene product was the target gene product encoding only the heavy chain of human ferritin, and the sequence was shown below:

ATGACGACCGCGTCCACCTCGCAGGTGCGCCAGAACTACCACCAGGACTC
AGAGGCCGCCATCAACCGCCAGATCAACCTGGAGCTCTACGCCTCCTACG
TTTACCTGTCCATGTCTTACTACTTTGACCGCGATGATGTGGCCTTGAAG
AACTTTGCCAAATACTTTCTTCACCAATCTCATGAGGAGAGGGAACATGC
TGAGAAACTGATGAAGCTGCAGAACCAACGAGGTGGCCGAATCTTCCTTC
AGGATATCAAGAAACCAGACTGTGATGACTGGGAGAGCGGGCTGAATGCG
ATGGAGTGTGCATTACATTTGGAAAAAAATGTGAATCAGTCACTACTGGA
ACTGCACAAACTGGCCACTGACAAAAATGACCCCCATTTGTGTGACTTCA
TTGAGACACATTACCTGAATGAGCAGGTGAAAGCCATCAAAGAATTGGGT
GACCACGTGACCAACTTGCGCAAGATGGGAGCGCCCGAATCCGGCTTGGC
GGAATATCTCTTTGACAAGCACACCCTGGGAGACAGTGATAATGAAAGCT
AG.

Subsequently, the amplified PCR product and pET-12b plasmid (Novagen, Inc., Madison, Wis., USA) were digested with the restriction enzymes NdeI and BamHI. The digested PCR product was ligated to pET-12b vector to produce the recombinant plasmid pET12b-HFn containing the gene sequence of only the heavy chain subunit of human ferritin. The map of the plasmid was shown in the figure below, in which the enzyme sites were marked with red arrows, and the antibiotics resistance was Amp.

The resultant plasmid pET12b-HFn was used to transfect an expression strain, BL21DE3 (commercially available from Beijing Transgen Biotech. Co. Ltd, China) to express the heavy chain subunit of ferritin. The bacteria culture expressing the target protein was disrupted under ultrasound, and the debris of E. coli was removed by centrifugation. The supernatant was heated at 65° C. for 10 min. The impurity proteins were precipitated and removed by centrifugation. The resultant supernatant was separated and purified on the exclusion chromatography Sepharose 4B column to produce the recombinant heavy chain subunit of ferritin, which was tested for purity by electrophoresis and for protein concentration by BCA assay.

The biomineralization of the bionic ferritin was briefly described as follows:

100 mM of sodium chloride solution was subjected to degasification treatment, and was put into a sealed reactor full of nitrogen gas together with the purified ferritin protein shell. The reaction temperature was maintained at 65° C., pH was kept at 8.5. (NH₄)₂Fe(SO₄)₂.6H₂O was added at the ratio of 1000-5000 Fe ions/ferritin shell molecule. Hydrogen peroxide, as an oxidant, was added together with Fe ion at a ratio of H₂O₂:Fe²⁺=1:3. After the addition of hydrogen peroxide and Fe ion, the reaction was kept for 5 min 200 μl of 300 mM sodium citrate was added to complex the remaining Fe ion. The principle for generating magnetic bionic ferritin was shown in Formula (1) listed below. The product was collected and purified via exclusion chromatography. The denatured impurity proteins were removed and the magnetized bionic ferritin consisting of only the heavy chain subunit was obtained.

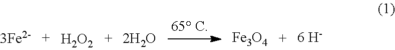

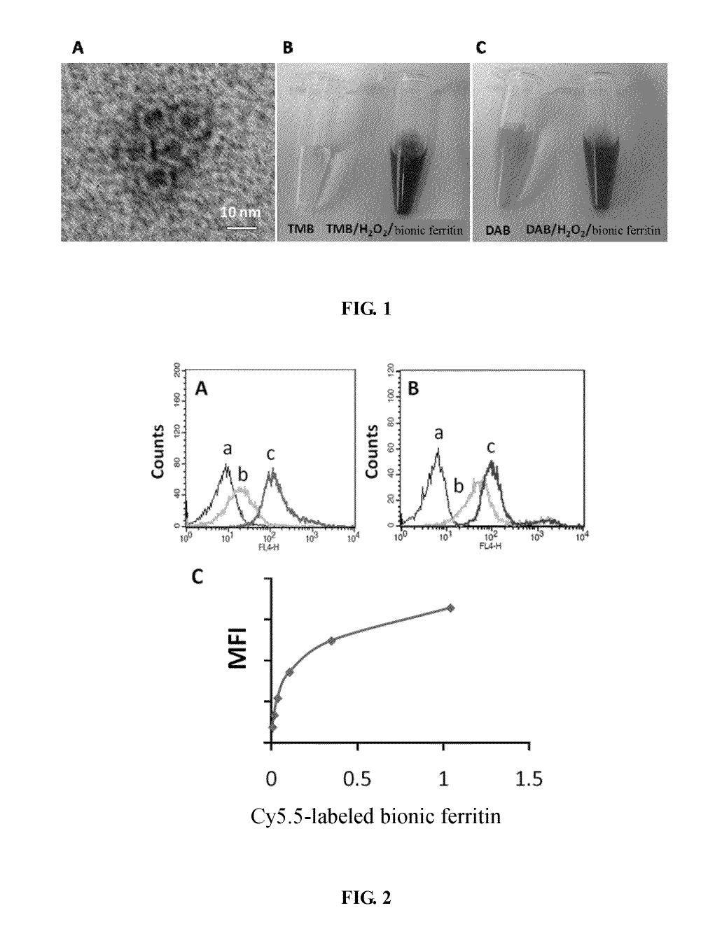

FIG. 1A was the TEM photos of the synthesized bionic ferritin. From this figure, it could be seen that the bionic ferritin was dispersed homogeneously; the outer diameter of the protein shell was about 12 nm, and the inner diameter of the protein shell was about 8 nm

### Example 2

### The Peroxidase Activity of the Bionic Ferritin

According to the report of our research group, the magnetic iron oxide nanoparticles had a similar catalytic activity to a peroxidase (Gao L, et al. (2007) Nature Nanotech., 2: 577-583.). Because the bionic ferritin enclosed an iron oxide core, it is supposed to have a peroxidase-like activity. We tested the enzyme activity of the bionic ferritin with a substrate of HRP. The assay was specifically described as follows: adding 30% of H₂O₂ and TMB or DAB in the bionic ferritin to observe the change of color. As shown in FIG. 1B, after the bionic ferritin and H₂O₂ were added to TMB, it turned to dark blue, and after the bionic ferritin and H₂O₂ were added to DAB, it turned to dark brown. The color reaction was the same as that of HRP, indicating that the bionic ferritin had a similar peroxidase activity.

### Example 3

### The Bionic Ferritin Binds to Tumor Cells

In order to investigate the binding of the bionic ferritin to human tumor cells, the common human tumor cells were selected to incubate with the bionic ferritin labeled with a fluorescent molecule, and the binding of the bionic ferritin to each kind of tumor cells was detected by flow cytometry.

The experimental method was as follows: according to the labeling method provided in the instructions, the bionic ferritin was labeled with NHS-activated Cy5.5 (Cy5.5-NHS, GE Healthcare); each strain of cells was cultured to the density of about 1×10⁵, and digested with trypsin; the cells were washed with 0.3% of BSA/PBS for three times; 50 μg/ml Cy5.5-labeled bionic ferritin was added and incubated at 4° C. for 45 min; and then, the cells were washed with 0.3% of BSA/PBS for three times, and finally resuspended in PBS; the cell sample was tested for fluorescence via flow cytometry.

The result was shown in Table 1. The bionic ferritin had reaction activity with 11 cell strains of 12 cell strains to be tested, which were the cells of hepatocellular carcinoma, colorectal cancer, breast cancer, melanoma, monocyte lymphoma, cervix cancer, leukemia and prostate cancer, respectively, indicating that the bionic ferritin was capable of specifically binding to the majority of cancer cells.

TABLE 1
The analysis of the reaction activity of the bionic
ferritin with human tumor cells via flow cytometry.

| Cell line/catalog No. | Tumor type | reactivity |
|---|---|---|
| SMMC-7721/(TCHu 52) | hepatocellular carcinoma | + |
| HT29/(HTB-38) | colorectal cancer | + |
| MDA-MB-231/(HTB-26) | breast cancer | + |
| MX-1/(KG038) | breast cancer | − |
| A375/(CRL-1619) | melanoma | + |
| K562/(CCL-243) | human chronic myelogenous | + |
| | leukemia | |
| HeLa/(CCL-2) | cervix cancer | + |
| SKOV3/(HTB-77) | ovary cancer | + |
| PC-3/(CRL-1435) | prostate cancer | + |
| U251/(TCHu 58) | glioma | + |
| U937/(CRL-1593.2) | lymphoma | + |
| Jurkatr/(TIB-152) | leukemia T cells | + |

* SMMC-7721 and U251 were commercially available from Chinese Academy of Science Type Culture Collection Cell Bank, MX-1 was commercially available from KeyGen Biotech. Co. Ltd, and the others were commercially available from ATCC.

### Example 4

### The Interaction of the Cancer Cells with the Bionic Ferritin

In order to investigate the binding of the bionic ferritin to the tumor cell surface receptors, the inventor incubated different concentrations of Cy5.5-labeled bionic ferritin with colorectal cancer cell HT29 to obtain the binding saturation curve of bionic ferritin. The receptor sites were blocked with excessive anti-TfR1 antibody to demonstrate that TfR1 mediated the interaction of bionic ferritin with cancer cells.

The experimental method was as follows: the cancer cells HT29 were cultured to the density of about 1×10⁵, and digested with trypsin; the cells were washed with 0.3% of BSA/PBS for three times; different concentrations of Cy5.5-labeled bionic ferritin were added and incubated at 4° C. for 45 min; and then, the cells were washed with 0.3% of BSA/PBS for three times, and finally resuspended in PBS; the cell sample was tested for fluorescence via flow cytometry. A saturation curve was plotted based on the fluorescence intensity and the concentration of the bionic ferritin. The result was shown in FIG. 2C. The binding of the bionic ferritin to tumor cells is saturable, indicating that such binding was specific and had a high affinity.

Subsequently, based on the saturation curve, a suitable concentration of Cy5.5-labeled bionic ferritin was selected for the competition binding inhibition assay. Likewise, after the cells were cultured to the density of about 1×10⁵, digested with trypsin, and washed with buffers, a certain concentration of Cy5.5-labeled bionic ferritin and excessive unlabeled bionic ferritin were added to the cells at the same time, and incubated at 4° C. for 45 min. The cells were washed for three times, and finally resuspended in PBS. The cell sample was tested for fluorescence via flow cytometry. The result was shown in FIG. 2A. The labeled bionic ferritin bound to the cells, but majority of them could be competed with excessive unlabeled bionic ferritin, further indicating that the binding of bionic ferritin to cancer cells was specific, with only a small number of non-specific binding molecules left (not being competed) on the cell surface.

Li L. et al., (2010) Proc. Natl. Acad. Sci. 107: 3505-3510, reported the co-receptor of human ferritin and transferrin, TfR1. In order to demonstrate that TfR1 mediates the interaction of bionic ferritin with cancer cells, we used excessive anti-TfR1 antibody to compete with Cy5.5-labeled bionic ferritin for the cell surface receptor. The specific experimental method was as follows: HT29 cells were cultured to the density of about 1×10⁵, and digested with trypsin; the cells were washed for three times; excessive mice anti-human TfR1 antibody CD71 (BD Pharmingen) and Cy5.5-labeled bionic ferritin were added at the same time and incubated at 4° C. for 45 min; the cells were washed with 0.3% of BSA/PBS for three times, and finally resuspended in PBS; the cell sample was tested for fluorescence via flow cytometry.

The result was shown in FIG. 2B. About 50% of the bionic ferritin was competed, indicating that TfR1 mediated the interaction of the bionic ferritin with cancer cells.

### Example 5

### Specific Detection of an Implanted Tumor in Mice Using the Bionic Ferritin

In order to investigate the recognition and staining capacity of the bionic ferritin to tumor tissues, we incubated it with the nude mice-implanted tumor tissue, and compared with the result of conventional immunohistochemistry method using antibodies.

The specific experimental method was as follows:

The paraffin section of mice-implanted tumor was made by the following method: the cultured HT-29, SMMC-7721 and SKOV-3 tumor cells (ATCC) were collected, washed with serum free medium, and resuspended in PBS buffer. The female BALB/c mice (Beijing Experimental Animal Center, China) were subcutaneously injected with 5×10⁶ HT-29, SMMC-7721 or SKOV-3 cells at shoulder site. The mice were sacrificed when the tumor grew to 0.4 to 0.6 cm in diameter, and the tumor was taken and fixed with 4% of paraformaldehyde for 12 to 24 hours, washed with 70% of ethanol, and gradually dehydrated with 70-80-90-95-95-100% of ethanol for 35 min, immersed into 1:1 ethanol/xylene solution for 5 min, taken from ethanol/xylene solution and permeabilized twice by immersing into xylene for 20 min, put into hot-molten paraffin (melting point of 56-58° C.) for 4 hours. The wax-dipped tissue was taken and transferred to a paper box containing molten paraffin, with the incisal surface downwards. It was embedded after the surface of paraffin was coagulated, and solidified by immersing into cold water to produce a paraffin block. The paraffin-embedded tissue was cut into sections with 5 μm thickness by Leica paraffin slicer. The section was spread on a slide at 42° C., dried at room temperature and baked at 50° C. for 6 hours to produce the paraffin sections of mice-implanted HT-29, SMMC-7721 and SKOV-3 tumors, respectively.

The method for staining tumor-implanted tissues with anti-TfR1 antibody CD71 was briefly described as follows: the paraffin sections of the implanted tumor were deparaffinaged to water, washed with PBS for three times, incubated for 30 min by adding methanol solution containing 0.3% of H₂O₂ to inactivate the endogenous peroxidase in the tissue; 1 mM citric acid antigen restoration solution was added, and heated in microwave oven for 30 min for antigen restoration; after rinsed with deionized water, it was blocked with 5% of ordinary sheep serum at 37° C. for 1 hour; Anti-TfR1 antibody CD71 (BD Pharmingen) was added and incubated at 4° C. over night; after rinsed with PBS, 1000-fold diluted secondary antibody was added and incubated at 37° C. for 1 hour, and then rinsed with PBS; 2000-fold diluted HRP-labeled tertiary antibody was added and incubated at 37° C. for 1 hour, and then rinsed with PBS; one droplet of each of Buffer, H₂O₂ and DAB contained in DAB kit was added into 1 ml deionized water, mixed uniformly and dropped onto the tissue specimen on the slide to develop for 5 min; it was rinsed with deionized water, counter-stained with haematoxylin, dehydrated and mounted, and then observed with microscope for imagining.

The method for staining mice-implanted tumor tissue with bionic ferritin was briefly described as follows: after deparaffinage treatment of the paraffin sections of the implanted tumor, inactivating of the endogenous peroxidase in the tissue, restoring of the antigen, as well as blocking with the serum, the bionic ferritin was added, and incubated at room temperature for 45 min, the specimen was rinsed with PBS, then stained with DAB, counter-stained with haematoxylin, conventionally dehydrated and mounted, and then observed under microscope.

Staining Mice-Implanted Tumor with FITC Labeled Bionic Ferritin:

According to the method recorded in the instructions, the bionic ferritin was labeled with fluorescein isothiocyanate FITC (Sigma-Aldrich). After the paraffin section of the implanted tumor tissue was blocked with serum, the FITC labeled bionic ferritin was added and incubated at room temperature for 45 min, rinsed twice with PBS, and the nuclei were stained with DAPI for 10 min. The quencher was added and the section was mounted and observed under fluorescent confocal microscopy.

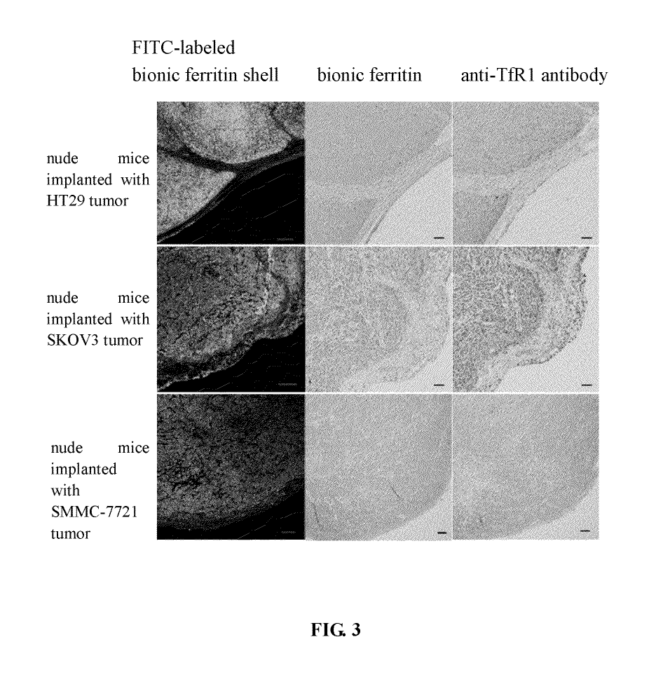

The result was shown in FIG. 3. The bionic ferritin was capable of specifically recognizing the tumor cells in the tissues. The stained region was consistent with that of antibody CD71. The staining effect of the tumors was comparable to that of the antibody. The change of cell morphology could be clearly distinguished. The fluorescent confocal result which showing the recognition of tumor tissues by the bionic ferritin further demonstrated that the bionic ferritin shell can achieve the specific recognition of a tumor. As compared with the conventional immunohistochemistry method, the bionic ferritin can achieve the specific recognition for the antigen and the staining in one step, without the need of multiple steps of incubation with a primary antibody, a secondary antibody and a tertiary antibody, and of modification with a signal molecule such as HRP enzyme and the like. And thus, it is simple, convenient and quick, as compared with the conventional immunohistochemistry method.

### Example 6

### The Bionic Ferritin Recognizes and Stains the Clinic Human Liver Cancer Tissues

Liver tissue specimens were obtained from Beijing Anzhen Hospital and Beijing Tumor Hospital. All the liver tissue specimens were the bulk specimens of the surgically ablated tumor or adjacent tissues. All the tissues were embedded in paraffin.

The method for staining the tissue sections with bionic ferritin was briefly described as follows: after deparaffinage treatment of the paraffin-embedded liver cancer tissue and normal liver tissue, inactivating of the endogenous peroxidase in the tissue, restoring of the antigen, as well as blocking with the serum, the bionic ferritin was added, and incubated at room temperature for 45 min, the specimen was rinsed with PBS, then stained with DAB, counter-stained with haematoxylin, conventionally dehydrated and mounted, and then observed under microscope.

The method for fluorescent staining tissue sections with bionic ferritin was briefly described as follows: after deparaffinage treatment of the paraffin-embedded liver cancer tissue and normal liver tissue as well as blocking with serum, the FITC-labeled bionic ferritin shell was added and incubated at room temperature for 45 min, the specimen was rinsed twice with PBS, and the nuclei were stained with DAPI for 10 min; the quencher was added and the section was mounted and observed under fluorescent confocal microscopy.

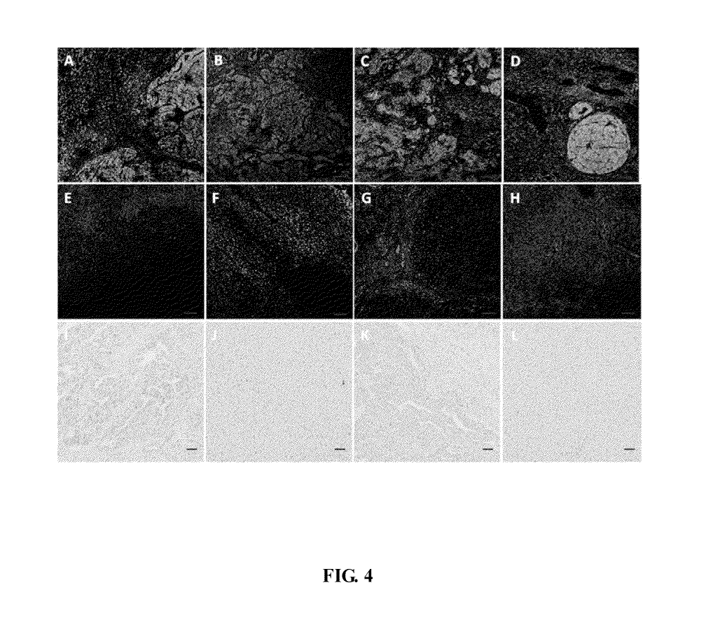

The result was shown in FIG. 4. The bionic ferritin was capable of specifically recognizing the cancerous cells in liver tissue, and it did not stain the normal tissue adjacent to the tumor, and did not recognize the tissues such as chronic inflammation of liver tissue, nodular cirrhosis, biliary cirrhosis and the like. In all the tested hepatocellular carcinoma tissues, the positive ratio was 54/55, while the positive ratio in the normal liver tissue, hepatitis and cirrhosis was 7/45, indicating that the bionic ferritin was capable of specifically recognizing liver cancer tissues.

### Example 7

### The Bionic Ferritin Specifically Recognizes Various Human Cancer Tissues

In order to systemically investigate the capacity of bionic ferritin to specifically recognize the cancerous tissues and human normal tissues, we selected paraffin section specimens of human tumor tissues and 48 cases of human normal tissues or inflammation tissues for bionic ferritin staining. The patient tissue specimens were obtained from Beijing Anzhen Hospital, Beijing Tumor Hospital and Xi'an Aomei Biotech. Co. Ltd. All the liver tissue specimens were the bulk specimens of the surgically ablated tumor or adjacent tissues. All the tissues were embedded in paraffin.

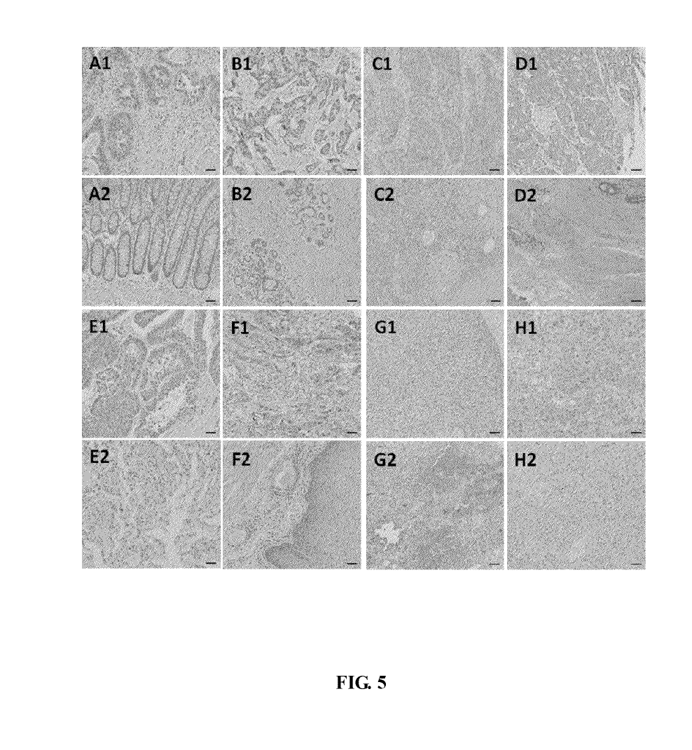

The method for staining the tissue sections with bionic ferritin was briefly described as follows: after deparaffinage treatment of the paraffin-embedded human tumor tissue, normal tissue or related inflammation tissue, inactivating of the endogenous peroxidase in the tissue restoring of the antigen, as well as blocking with serum, the bionic ferritin was added, and incubated at room temperature for 45 min, the specimen was rinsed with PBS, stained with DAB, counter-stained with haematoxylin, conventionally dehydrated and mounted, and then observed under microscope. The tissue-recognition results were shown in FIG. 5 and Table 2.

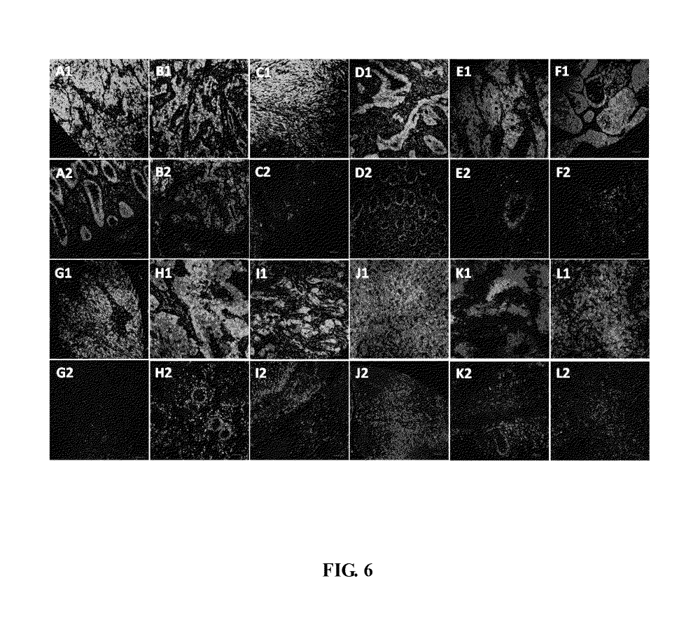

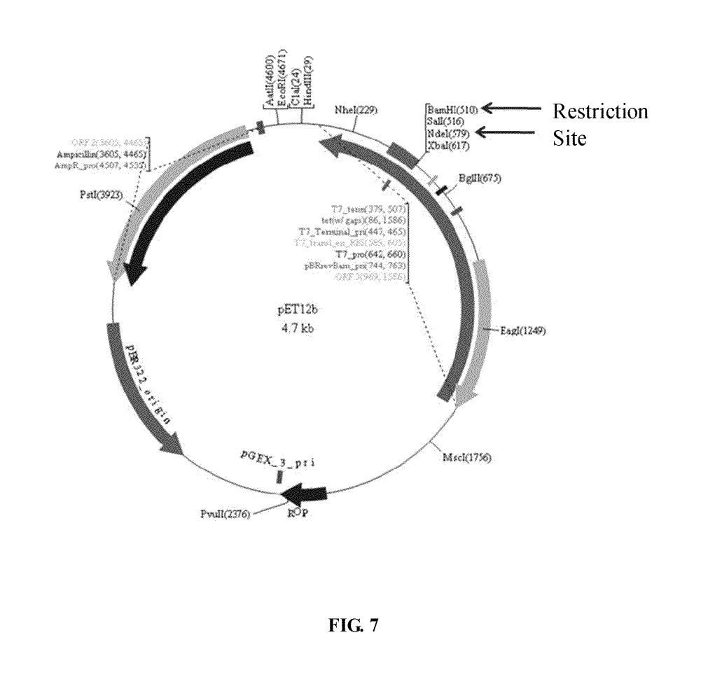

The method for fluorescent staining tissue sections with bionic ferritin was briefly described as follows: after deparaffinage treatment of the paraffin-embedded tumor tissue, normal tissue or inflammation tissue as well as blocking with serum, the FITC-labeled bionic ferritin shell was added and incubated at room temperature for 45 min, the specimen was rinsed twice with PBS, and the nuclei were stained with DAPI for 10 min; the quencher was added and the section was mounted and observed under fluorescent confocal microscopy. The result was shown in FIG. 6.

As shown in FIGS. 5 and 6, the bionic ferritin exhibited strong binding to colon cancer, breast cancer, ovary cancer, colon adenocarcinoma, pulmonary squamous cell carcinoma, prostate cancer, esophagus squamous cell carcinoma, thymic carcinoma and cervix squamous cell carcinoma, and like, while it exhibited no relative binding to the corresponding chronic colon inflammation, breast tissue, ovary tissue, colon mucous membrane tissue, lung tissue, prostate proliferation, chronic esophagus inflammation, thymic tissue, cervix tissue and chronic cervix inflammation and the like. The analysis result showed in Table 2 indicated that the bionic ferritin exhibited excellent staining sensitivity and specificity to hepatocellular carcinoma (54/55), pulmonary squamous cell carcinoma (50/52), thymic carcinoma (4/4), cervix squamous cell carcinoma (4/4), esophagus cancer (3/3), colon cancer (6/6) and ovary cancer (4/4) and the like. The tissue-recognition effect shown in FIGS. 5 and 6 demonstrated that the bionic ferritin is capable of specifically recognizing various human cancerous tissues, especially strongly staining and recognizing hepatocellular carcinoma, lung cancer, thymic carcinoma, cervix cancer, ovary cancer and colon cancer, while it exhibited no recognition to the corresponding normal or inflammation tissues. As compared with the conventional immunohistochemistry method, the method for identifying cancerous tissues based on bionic ferritin is capable of specifically recognizing tumors and staining in one step, without the need of multiple steps of incubation with a primary antibody, a secondary antibody and a tertiary antibody, and of labeling with an enzyme such as HRP etc. or a signal molecule, the manipulation thereof is simple and moreover, the staining effect thereof to the tumor tissues is comparable to that of the immunohistochemistry method.

TABLE 2
The analysis of the specific recognition of bionic ferritin to
clinical tumor tissues and the corresponding normal tissues.

| | Positive | | Positive |
|---|---|---|---|
| | cases/total | Normal tissues/inflammation | cases/total |
| Tumor tissues | cases | tissues | cases |
| hepatocellular | 54/55 | normal liver/hepatitis/cirrhosis | 7/45 |
| carcinoma | | | |
| pulmonary | 50/52 | pulmonary tissue | 1/56 |
| squamous cell | | | |
| carcinoma | | | |
| thymic carcinoma | 4/4 | thymic tissue | 0/4 |
| cervix squamous | 28/28 | cervix tissue/chronic | 0/52 |
| cell carcinoma | | inflammation | |
| prostate cancer | 22/22 | prostate smooth muscle/ | 2/16 |
| | | prostate proliferation | |
| mammary | 4/5 | breast tissue/breast fibrosis/ | 1/4 |
| non-specific | | proliferative fibrous tissue/ | |
| infiltrative duct | | breast adipose tissue | |
| carcinoma | | | |
| ovary cancer | 55/56 | ovary tissue/ovary epithelial | 1/16 |
| | | proliferation | |
| esophagus | 3/3 | esophagus smooth | 0/5 |
| squamous cell | | muscle/chronic inflammation | |
| cancer | | companied with epithelial | |
| | | proliferation | |
| colon | 23/23 | colon mucous membrane | 1/41 |
| adenocarcinoma | | tissue/chronic inflammation | |
| | | of colon mucous membrane | |

### REFERENCES

1. Shi S R., (2008), Evaluation of the value of frozen tissue section used as “Gold Standard” for immunohistochemistry, Am. J. Clin. Pathol., 129:358-366.
2. Taylor C R., (2006), Standardization in immunohistochemistry: the role of antigen retrieval in molecular morphology, Biotech Histochem., 81:3-12.
3. Larsson L., (1988), Immunocytochemistry: Theory and Practice. Boca Raton, Fla.: CRC Press, 41-73.
4. Turyanska L., et al. (2009), The Biocompatibility of Apoferritin-Encapsulated PbS Quantum Dots, Small, 5:1738-1741.
5. Meldrum F C., et al. (2009), Magnetoferritin:in vitro synthesis of a novel magnetic protein, Science, 257:522-523.
6. Galvez N., et al. (2006), Apoferritin-encapsulated Ni and Co superparamagnetic nanoparticles, J. Mater. Chem., 16:2757-2761.
7. Hennequin B. et al. (2008), Aqueous near-infrared fluorescent composites based on apoferritin-encapsulated PbS quantum dots, Adv. Mater., 20:3592-3593.
8. Yamashita I., et al. (2004), Bio-template synthesis of uniform CdSe nanoparticles using cage-shaped protein, apoferritin, Chem. Lett., 33:1158-1159.
9. Uchida M., et al. (2006), Targeting of Cancer Cells with Ferrimagnetic ferritin cage nanoparticles, J. Am. Chem. Soc., 128: 16626-16633.
10. Lin X., et al. (2011), Chimeric ferritin nanocages for multiple function loading and multimodal imaging, Nano Lett., 11: 814-819,
11. Geninatti C S., et al. (2006) Magnetic resonance visualization of tumor angiogenesis by targeting neural cell adhesion molecules with the highly sensitive gadolinium-loaded apoferritin probe, Cancer Res., 66: 9196-201.
12. Gao L., et al. (2007), Intrinsic peroxidase-like activity of ferromagnetic nanoparticles, Nature Nanotech., 2:577-583.
13. Chinese Patent No. 200610057413.9
14. Theil E C., (1987), Ferritin: structure, gene regulation, and cellular function in animals, plants, and microorganisms. Annu. Rev. Biochem., 56:289-315.
15. Harrison P M., et al. (1996) The ferritins: molecular properties, iron storage function and cellular regulation, Biochim. Biophys. Acta, 1275:161-203.
16. Levi S., et al. (1988), Mechanism of ferritin iron uptake—Activity of the H-chain and deletion mapping of the ferro-oxidase site—A study of iron uptake and ferro-oxidase activity of human-liver, recombinant H-chain ferritins, and of 2H-chain deletion mutants, J. Biol. Chem., 263:18086-18092.
17. Ford G C., et al. (1984), Ferritin: Design and formation of an iron-storage molecule, Philos Trans. R. Soc. Lond B Biol. Sci., 304:551-565.
18. Fargion S, et al. (1988) Characteristics and expression of binding sites specific for ferritin H-chain on human cell lines. Blood 71:753-757.
19. Moss D, et al. (1992) Characterization of the ferritin receptors of human T lymphoid (MOLT-4) cells, J. Lab Clin. Med. 119:273-279.
20. Bretscher M S, et al. (1983) Distribution of ferritin receptors and coated pits on giant HeLa cells, EMBO J. 2:599-603.
21. Takami M, et al. (1986) Human placental ferritin receptor. Biochim. Biophys Acta 884:31-38.
22. Fargion S, et al. (1991) Specific binding sites for H-ferritin on human lymphocytes: Modulation during cellular proliferation and potential implication in cell growth control, Blood 78:1056-1061.
23. Moss D., et al. (1992) Functional roles of the ferritin receptors of human liver, hepatoma, lymphoid and erythroid cells, J. Inorg. Biochem. 47:219-227.
24. Li L., et al. (2010), Binding and uptake of H-ferritin are mediated by human transferrin receptor-1, Proc. Natl. Acad Sci. USA, 107: 3505-3510.
25. Larrick J W, et al. (1979), Modulation of cell surface iron transferrin receptors by cellular density and state of activation, J. Supramol. Struct., 11:579-586.
26. Daniels T R., et al. (2006), The transferrin receptor part I: biology and targeting with cytotoxic antibodies for the treatment of cancer, Clin. Immunol., 121: 144-158.
27. Ryschich E., et al. (2004), Transferrin receptor is a marker of malignant phenotype in human pancreatic cancer and in neuroendocrine carcinoma of the pancreas, Eur. J. Caner, 40:1418-1422.
28. Santambrogio P., et al. (2000), Functional and immunological analysis of recombinant mouse H- and L-ferritins from Escherichia coli, Protein Expr. Purif., 19: 212-218.

1. A bifunctional tumor diagnostic reagent, characterized in that it consists of a protein shell for specifically recognizing cancer tissues and/or cancer cells and an inorganic nano-core having the catalytic activity of a peroxidase.

2. The bifunctional tumor diagnostic reagent according to claim 1, characterized in that the protein shell comprises a protein shell having a nano-cavity structure such as apoferritin, heat shock protein, DNA-binding protein, pteridine synthase, and a viral protein shell having a nano-cavity structure, wherein the viral protein shell having a nano-cavity structure comprises cowpea chlorotic mottle virus and cowpea mosaic virus protein.

3. The bifunctional tumor diagnostic reagent according to claim 2, characterized in that the apoferritin is self-assembled from 12 or 24 heavy chain subunits and light chain subunits at any ratio.

4. The bifunctional tumor diagnostic reagent according to claim 3, characterized in that the apoferritin is a natural apoferritin or genetically recombinant apoferritin, wherein the natural apoferritin is derived from an eukaryotic or a prokaryotic organism, preferably from a mammal.

5. The bifunctional tumor diagnostic reagent according to claim 1, characterized in that the protein shell comprises a protein shell chemically coupled or genetically fused to a targeting molecule and/or a signal molecule, wherein the targeting molecule comprises a polypeptide or a nucleic acid aptamer, and wherein the polypeptide comprises an antibody, and wherein the signal molecule comprises a fluorescent molecule, a radioactive nuclide and an enzyme.

6. The bifunctional tumor diagnostic reagent according to claim 1, characterized in that the inorganic nano-core comprises a magnetic iron oxide nanoparticle, an iron sulfide nanoparticle, a noble metal-blended iron oxide nanoparticle, a double-metal alloy nanoparticle, and a cerium oxide nanoparticle.

7. The bifunctional tumor diagnostic reagent according to claim 1, characterized in that the protein shell is a generically recombinant ferritin, consisting of heavy chain subunits, and the inorganic core is a magnetic iron oxide nanoparticle.

8. A kit for tumor diagnosis, comprising the bifunctional tumor diagnostic reagent according to claim 1 and a substrate of a peroxidase.

9. A bionic ferritin and the use thereof in the manufacture of a tumor diagnostic reagent, wherein the bionic ferritin consists of a protein shell and an inorganic nano-core, wherein the protein shell is a generically recombinant ferritin consisting of heavy chain subunits, and the inorganic nano-core is a magnetic iron oxide nanoparticle.

10. A method of detecting a tumor in an individual, comprising, obtaining a body tissue or a cell sample from said individual, contacting said sample with the bifunctional tumor diagnostic reagent according to claim 1, adding a substrate for direct development, thereby determining the presence of a tumor in said individual, preferably, said tissue is a puncture biopsy specimen, a postoperative pathological section or an autopsy tissue from a clinical patient or an animal model, and said cell is an exfoliated cell in the circulatory system of an organism, a tissue lytic cell or an in vitro cultured cell.


SEQUENCE LISTING (3 sequences)

SEQ ID NO: 1 - DNA, 552 bp, Homo sapiens
atgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggccttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcgatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatccggcttggcggaatatctctttgacaagcacaccctgggagacagtgataatgaaagctag

SEQ ID NO: 2 - DNA, 25 bp, Artificial sequence
  Synthetic oligonucleotide
agtcgcccatatgacgaccgcgtcc

SEQ ID NO: 3 - DNA, 26 bp, Artificial sequence
  Synthetic oligonucleotide
gccggatccttagctttcattatcac